(19) United States
(12) Patent Application Publication
(10) Pub. No.: US 2025/0276943 A1
(43) Pub. Date: Sep. 4, 2025

(54) METHOD FOR PROMOTING THE GROWTH OF POLYPORUS UMBELLATUS MYCELIUM, RAOULTELLA ORNITHINOLYTICA OF GENUS RAOULTELLA CONTAINED THEREIN AND USE THEREOF

(71) Applicant: Institute of TCM Health Industry CACMS, Nanchang (CN)

(72) Inventors: Tianrui LIU, Nanchang (CN); Yuan YUAN, Nanchang (CN); Rui CHENG, Nanchang (CN); Hui LI, Nanchang (CN); Haiyun GAO, Nanchang (CN); Yao LI, Nanchang (CN); Yuxin CHEN, Nanchang (CN)

(21) Appl. No.: 18/959,945

(22) Filed: Nov. 26, 2024

(30) Foreign Application Priority Data
Mar. 4, 2024 (CN) 202410242290.4

(51) Int. Cl. C05F 11/08; C12N 1/20; C12R 1/01

(52) U.S. Cl. CPC C05F 11/08; C12N 1/205; C12R 2001/01

(57) ABSTRACT

A method for promoting the growth of Polyporus umbellatus (PU) mycelium is provided, Raoultella ornithinolytica (R. ornithinolytica) of genus Raoultella contained therein and use thereof are provided, belonging to the field of biotechnology. The growth of PU mycelium is promoted by administering to the PU mycelium R. ornithinolytica wherein the Raoultella ornithinolytica is a R. ornithinolytica strain having a strain number of FL19, and registratered under CGMCC No. 29044 in the China General Microbiological Culture Collection Center. The R. ornithinolytica FL19 of genus Raoultella provided by the present disclosure may promotes the growth of PU mycelium, and also solubilize phosphate and produce siderophore and auxin.

### CROSS-REFERENCE TO RELATED APPLICATION

This patent application claims the benefit and priority of Chinese Patent Application No. 202410242290.4 filed with the China National Intellectual Property Administration on Mar. 4, 2024, the disclosure of which is incorporated by reference herein in its entirety as part of the present application.

### REFERENCE TO SEQUENCE LISTING

A computer readable XML file entitled “GWP20240906745_seqlist”, that was created on Nov. 8, 2024, with a file size of about 9,385 bytes, contains the sequence listing for this application, has been filed with this application, and is hereby incorporated by reference in its entirety.

### TECHNICAL FIELD

The present disclosure belongs to the field of biotechnology, and in particular relates to a method for promoting the growth of Polyporus umbellatus mycelium, Raoultella ornithinolytica of genus Raoultella contained therein and use thereof.

### BACKGROUND

Polyporus umbellatus (Pers.)Fries. is a medicinal fungus of Polyporus in the family Polyporaceae, and its sclerotia have been used as medicine for more than 2500 years. Polyporus umbellatus (PU) is first described in “Shennong's Classic of Materia Medica”. It is neutral in nature, sweet and thin in taste, and it is associated with spleen, liver and bladder meridians. It has the effects of removing dampness and promoting diuresis, etc.^{[1]}. PU is named because of its black sclerotium epidermis and the shape of shrunken and nodulous protrusions similar to pig dung, also known as Zhushi Ling, Di Wutao and Zhu Fuling in Chinese. The active ingredients in PU mainly include polysaccharides, steroids, proteins, amino acids, vitamins and trace inorganic elements, etc.^{[3]}. Ergosterol, as an indicator ingredient of PU specified in the “Chinese Pharmacopoeia”, has a diuretic effect; and modern studies have shown that PU polysaccharides have the functions of resisting tumors, enhancing immune function, protecting liver, etc., and are also one of the important active ingredients in PU.

The growth of PU is inseparable from Armillaria mellea (Vahl) P. Kumm., which is a facultative parasitic fungus of Armillaria in the family Agaricaceae, order Agaricales, phylum Basidiomycota, and is widely distributed in temperate to tropical regions. During the cultivation of PU, Armillaria mellea infects Polyporus umbellatus sclerotia through the rhizomorph, and the resulting metabolites provide nutrients for the reproduction and growth of PU through the septate cavity and anti-invasion structure.

PU is mainly distributed in regions such as Shaanxi, Yunnan, Shanxi, Henan, Gansu, Sichuan, Jilin and Heilongjiang. Ningshan County, Ankang City, Shaanxi Province has become one of the important production areas of PU. At present, the cultivation of PU mainly adopts an imitated wild cultivation method, but problems such as long growth cycle, low yield, imperfect artificial cultivation technology, etc., have led to continuous rise in market prices of PU. With the rapid increase in the demand for the medicinal material PU, increasing the yield of PU, maintaining the stable content of active ingredients, and shortening the cultivation cycle of PU have become urgent issues in production. At present, research on PU mainly focuses on the biological characteristics, pharmacological activity, clinical application, quality standards, etc., while basic research on related the artificial cultivation technology of PU is less involved.

### SUMMARY

The technical problem to be solved by the present disclosure is to provide a method for promoting the growth of Polyporus umbellatus (PU) mycelium. The technical problem to be solved is not limited to the technical subject matter described, and other technical subject matters that are not mentioned herein will be clearly understood by a person skilled in the art from the following description.

In order to solve the above-mentioned technical problem, the present disclosure provides the following technical solutions.

The present disclosure provides a method for promoting the growth of PU mycelium, the method including administering to the PU mycelium Raoultella ornithinolytica (R. ornithinolytica), where the R. ornithinolytica is a R. ornithinolytica strain having a strain number of FL19, and deposited under CGMCC No. 29044 in the China General Microbiological Culture Collection Center.

The present disclosure also provides a R. ornithinolytica strain, where the strain has a strain number of FL19, and is deposited under CGMCC No. 29044 in the China General Microbiological Culture Collection Center.

The present disclosure also provides a culture of the Raoultella, where the culture is a substance obtained by culturing the Raoultella in a microorganism medium.

In the culture described above, the microorganism medium is a solid medium or a liquid medium.

The term “culture” refers to the liquid or solid products that contain a microbial population after artificial inoculation and culture (essentially all substances within a culture vessel, including fermentation products). These products are obtained by growing and/or proliferating microorganisms, which may be biologically pure cultures of the microorganisms, or may contain a certain amount of medium, metabolites and/or other ingredients produced during the culturing. The term “culture” also includes subcultures obtained by passaging microorganisms, which may be a culture of a certain passage or a mixture of cultures of several passages.

In some specific embodiments of the present disclosure, the microorganism medium may be LB medium.

The present disclosure also provides a microbial agent, which contains the aforementioned Raoultella or/and metabolites of the aforementioned Raoultella or/and the aforementioned culture.

In an embodiment, the microbial agent may also include a carrier. The carrier may be a solid carrier or a liquid carrier.

The solid carrier may be a mineral material, a biological material; the mineral material may be at least one of peat, clay, talc, kaolin, montmorillonite, white carbon, zeolite, silica and diatomaceous earth; and the biological material may be at least one of stalks of various crops, pine husks, rice straw, peanut shells, corn powder, soybean powder, starch, peat and animal manure. The liquid carrier may be water. In the microbial agent, R. ornithinolytica or/and metabolites of R. ornithinolytica may be present in the form of cultured living cells, the fermentation broth of living cells, the filtrate of a cell culture or a mixture of cells and filtrate. The dosage form of the microbial agent may include a variety of dosage forms, such as a liquid, an emulsion, a suspension, powder, granules, wettable powder or water-dispersible granules.

The present disclosure also provides use of the Raoultella, metabolites of the Raoultella, the culture, and the microbial agent in any one of:
A1) the promotion of growth of PU mycelium;
A2) the preparation of a product that promotes growth of PU mycelium;
A3) the promotion of growth of a plant;
A4) the preparation of a product that promotes growth of a plant;
A5) the production of siderophore;
A6) the preparation of a product that produces siderophore.

The R. ornithinolytica FL19 provided by the present disclosure may promote the growth of PU mycelium, and also solubilize phosphate and produce siderophore and auxin (IAA).

### Description of Deposit

Strain name: Raoultella ornithinolytica FL19
Latin name: Raoultella ornithinolytica
Strain number: FL19
Depository: China General Microbiological Culture Collection Center
Depository abbreviation: CGMCC
Address: No. 3, Yard 1, Beichen West Road, Chaoyang District, Beijing
Deposit date: Nov. 17, 2023
Deposit number: CGMCC No. 29044.

### BRIEF DESCRIPTION OF THE DRAWINGS

FIG. 1 shows the morphological characteristics of strain FL19;

FIG. 2 shows the phylogenetic analysis of strain FL19;

FIG. 3 shows the growth of PU mycelium;

FIG. 4 shows the growth of FL19 on PVK medium;

FIG. 5 shows the growth of FL19 on CAS medium;

FIG. 6 shows the ability of FL19 to produce indole-3-acetic acid (IAA) (Notes: on the left: medium that does not contain L-tryptophan; on the right: medium that contains L-tryptophan.);

FIG. 7 shows the IAA standard curve;

FIG. 8 shows the sequence of 16S rDNA of FL19 (SEQ ID NO: 1);

FIG. 9 shows the sequence of gyrB of FL19 (SEQ ID NO: 2).

### DETAILED DESCRIPTION OF THE EMBODIMENTS

The present disclosure is further described in detail below in conjunction with specific examples, and the examples given are only used to illustrate the present disclosure, but are not to limit the scope of the present disclosure. The examples provided below may serve as guidelines for further improvements by one of ordinary skill in the art and do not limit the present disclosure in any way.

Unless otherwise specified, the experimental methods in the following examples are conventional methods, and are performed according to techniques or conditions described in the literatures in the art or according to product instructions. The materials, reagents, etc. used in the following examples are all commercially available, unless otherwise specified.

### Example 1. Isolation, Screening and Identification of Strain FL19

### I. Isolation and Screening of the Strain

Preparation of solid medium: 10 g of peptone, 5 g of yeast powder, 10 g of sodium chloride and 15 g of agar were weighed, water was added to a final volume of 1 L, which was then autoclavined at 121° C. for 15 min. The liquid medium used during the experiment was a solid medium formula to which agar was not added.

Healthy PU cultivated for two years in Ningshan County, Shaanxi Province was collected and cultured using a traditional pure culture method. Fifty milliliters of sterile water was accurately measured to repeatedly rinse the soil attached to the surface of PU sclerotia until there was no soil attached to the surface. The collected rinsing liquid was placed in a 100 mL conical flask, and subjected to shaking culture on a shaker (25° C., 120 r/min) for 1 h, and then ultrasonicated at 160 W for 40 s.

One milliliter of soil suspension was taken and progressively diluted to 10-4 times, and 200 μL of the soil suspension diluted to 10-4 times was taken and plated on LB medium. After the surface of the medium was air-dried, the culture dish was placed invertedly in a thermostat at 25° C. for culture. The newly grown single colony was picked, transferred to 1 mL of the corresponding liquid medium, and then subjected to oscillation culture overnight on a constant temperature air bath shaker (200 rpm 25° C.). Isolation and purification were performed by performing three-quadrant streaking multiple times to obtain the pure culture of bacteria. Preliminary classification and deduplication were performed according to the morphology and characteristics of the bacteria on the medium, and the resulting bacteria were named as strain FL19.

### II. Identification of the Strain

### 1. Morphological Identification of the Strain

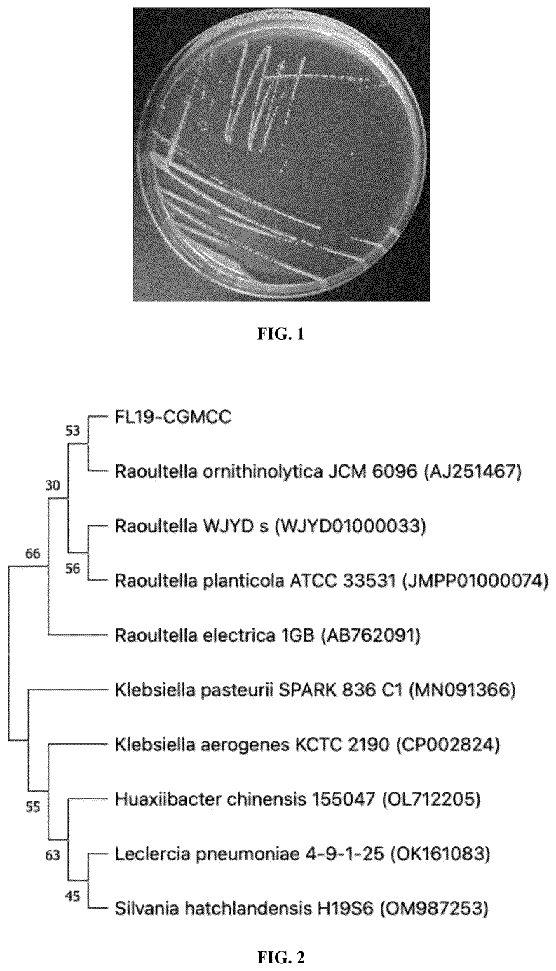

The morphological characteristics and growth characteristics of the bacteria were observed. The results showed that the strain was a gram-negative rod-shaped strain with a capsule, which formed larger round colonies when cultured on LB plates at 25° C. for 12 h (FIG. 1). The strain FL19 was negative for oxidase test and positive for catalase test (Table 1), which are consistent with the morphological characteristics and growth characteristics of the bacteria of genus Raoultella.

TABLE 1
Growth characteristics of strain FL19

| Test item | Results |
|---|---|
| Cell morphology | Rod-shaped |
| Gram staining | Negative |
| Catalase | + |
| Oxidase | − |

### 2. Physiological and Chemical Classification and Identification

Physiological and biochemical assays were performed on strain FL19. The results showed that strain FL19 utilized mannitol, D-glucose, salicin, D-melibiose, L-fucose, D-sorbitol, L-arabinose, citrate, histidine, 2-ketogluconate, 3-hydroxy-butyrate, 4-hydroxy-benzoate, L-proline, rhamnose, N-acetylglucosamine, D-ribose, inositol, sucrose, maltose, L-alanine, 5-keto-glucose hydrochloric acid and L-serine (Table 2), which is substantially consistent with the characteristics of the strain R. ornithinolytica of genus Raoultella.

TABLE 2
Strain growth experiment using API ID 32GN

| Test item | Results |
|---|---|
| Mannitol | − |
| D-glucose | − |
| Salicin | − |
| D-melibiose | − |
| L-fucose | − |
| D-sorbitol | − |
| L-arabinose | − |
| Propionate | + |
| Decanoate | + |
| Valerate | + |
| Citrate | + |
| Histidine | + |
| 2-ketogluconate | − |
| 3-hydroxy-butyrate | + |
| 4-hydroxy-benzoate | + |
| L-proline | + |
| Rhamnose | − |
| N-acetylglucosamine | − |
| D-ribose | + |
| Inositol | − |
| Sucrose | − |
| Maltose | − |
| Itaconic acid | − |
| Suberate | + |
| Malonate | + |
| Acetate | + |
| DL-lactate | + |
| L-alanine | + |
| 5-keto-glucose hydrochloric acid | − |
| Glycogen | − |
| 3-hydroxy-benzoate | + |
| L-serine | − |

### 3. Molecular Identification

The purified strain FL19 was inoculated into 500 μL of LB liquid medium, and then subjected to shaking culture on a shaker at 25° C. and 200 r/min for 12 h. The bacterial 16S rDNA (16S ribosomal DNA identification) fragment and DNA gyrase subunit B (gyrB) fragment in the bacterial liquid were amplified, respectively, to amplify partial sequences of the16S rDNA fragment and gyrB fragment, with the amplification primer sequence information as follows:

27F:
(SEQ ID NO: 3)
5′-AGAGTTTGATCCTGGCTCAG-3′;
1492R:
(SEQ ID NO: 4)
5′-TACGGCTACCTTGTTACGACTT-3′;
VP-F:
(SEQ ID NO: 5)
5′-AACGCGTTGTCWGARAAAGT-3′;
VP-R:
(SEQ ID NO: 6)
5′-CCTTTACGHCGRGTCATTTC-3′.

Amplification reaction system (25 μL): 2×M5 Super Fast Taq PCR Master Mix 12.5 μL; 1 μL each of reaction primers; 1 μL of bacterial liquid; ddH₂O made up to 25 μL. The PCR reaction procedure included pre-denaturation at 94° C. for 10 min, 35 cycles of denaturation at 94° C. for 30 s, annealing at 55° C. for 30 s, elongation at 72° C. for 1 min, and elongation at 72° C. for 10 min. Five microliters of the PCR product was taken and tested by electrophoresis using 1.0% agarose gel, and the PCR amplification product with a bright band at 1500 bp was sent to BGI Tech Solutions (Beijing Liuhe) Co., Ltd. for sequencing. The sequencing results of the 16S rDNA fragment of strain FL19 are as shown in FIG. 8:

The 16S rRNA gene sequence was sequenced, and aligned to that of bacteria of known type by EzBioCloud (www.ezbiocloud.net/eztaxon/). The results showed that the strain had the highest similarity to the known R. ornithinolytica JCM 6096, at 98.50% (Table 3).

TABLE 3
Alignment analysis of 16S rRNA gene sequence of strain FL19

| | | | | Pairwise | Mis- |
|---|---|---|---|---|---|
| | | | | Similarity | match/ |
| Rank | Name | Strain | Accession | (%) | Total nt |
| 1 | Raoultella | JCM 6096 | AJ251467 | 98.50 | 20/1337 |
| | ornithinolytica | | | | |
| 2 | WJYD s | RIT712 | WJYD01000033 | 98.21 | 24/1340 |
| 3 | Raoultella | ATCC | JMPP01000074 | 98.06 | 26/1340 |
| | planticola | 33531 | | | |
| 4 | Raoultella | 1GB | AB762091 | 97.91 | 28/1340 |
| | electrica | | | | |
| 5 | Klebsiella | KCTC | CP002824 | 97.76 | 30/1340 |
| | aerogenes | 2190 | | | |
| 6 | Huaxiibacter | 155047 | OL712205 | 97.61 | 32/1340 |
| | chinensis | | | | |
| 7 | Leclercia | 4-9-1-25 | OK161083 | 97.60 | 31/1292 |
| | pneumoniae | | | | |
| 8 | Silvania | H19S6 | OM987253 | 97.49 | 32/1276 |
| | hatchlandensis | | | | |
| 9 | Klebsiella | SPARK_ | MN091366 | 97.46 | 34/1340 |
| | pasteurii | 836_C1 | | | |
| 10 | Raoultella | ATCC | Y17658 | 97.46 | 34/1340 |
| | terrigena | 33257 | | | |

Pairwise Similarity: i.e., the similarity of 16S rRNA gene of strain FL19 to that of other strains.

The aforementioned 16S rDNA sequence of FL19 was subjected to alignment analysis by means of NCBI BLAST, the 16S rDNA sequences of homologous strains having high similarity were selected for phylogenetic analysis, and a phylogenetic tree was constructed using MEGA 11. The results are as shown in FIG. 2 (FL19-CGMCC in FIG. 2 represents strain FL19). It can be seen that strain FL19 and R. ornithinolytica JCM 6096 are clustered into the same branch.

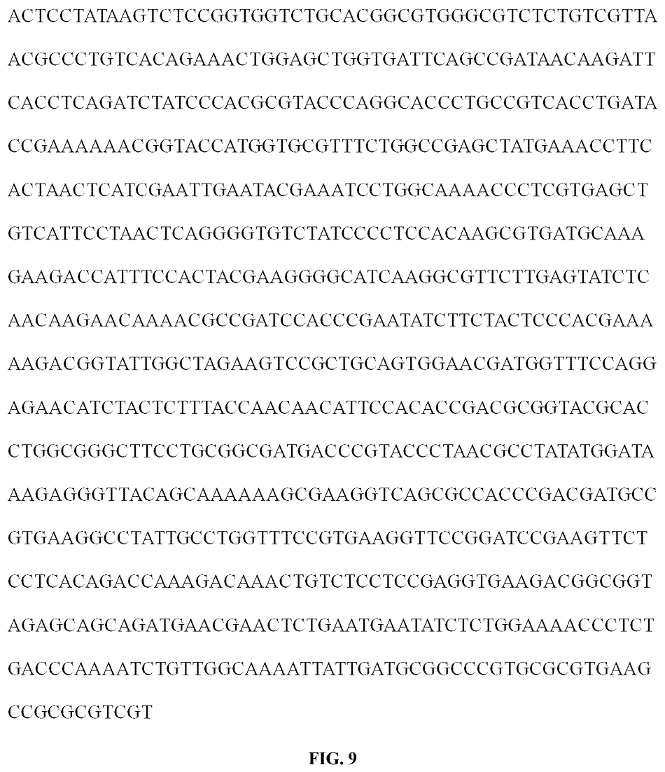

The gene sequence sequencing results of the gyrB fragment of strain FL19 are as shown in FIG. 9:

The gyrB gene sequence was sequenced, and alignment analysis was performed by means of NCBI BLAST. The results show that strain FL19 has the highest similarity to the strain R. ornithinolytica, at 94.54% (Table 4).

TABLE 4
Alignment analysis of gyrB gene sequence of strain FL19

| | | | | Pairwise |
|---|---|---|---|---|
| | | | | Similarity |
| Rank | Name | Strain | Accession | (%) |
| 1 | Raoultella | NCTC13096 | LR134195 | 94.54 |
| | ornithinolytica | | | |
| 2 | Raoultella | NCTC8846 | LR134230 | 94.54 |
| | ornithinolytica | | | |
| 3 | Raoultella | 24150 | CP138912 | 94.54 |
| | ornithinolytica | | | |
| 4 | Raoultella | GSH0205-8M-1 | AP019669 | 94.54 |
| | ornithinolytica | | | |
| 5 | Raoultella | OHMEATKPC2-NDM5 | CP054270 | 94.54 |
| | ornithinolytica | | | |

In view of the morphological characteristics of the colonies and bacteria, strain FL19 was identified as R. ornithinolytica of genus Raoultella, and was designated as R. ornithinolytica FL19. It can be referred to as R. ornithinolytica FL19 of genus Raoultella, or simply as strain FL19 or FL19.

### 4. Deposit of the Strain

In view of the above morphological characteristic analysis, physiological and biochemical characterization and 16S rDNA gene analysis results, strain FL19 was identified as R. ornithinolytica of genus Raoultella, with the designation FL19. The strain was deposited in the China General Microbiological Culture Collection Center (abbreviated as CGMCC; address: Institute of Microbiology, Chinese Academy of Sciences, No. 3, Yard 1, Beichen West Road, Chaoyang District, Beijing; zip code: 100101) with the deposit number of CGMCC No. 29044 on Dec. 17, 2023, hereinafter referred to as R. ornithinolytica FL19 of genus Raoultella or strain FL19 or FL19.

### Example 2. Analysis of Main Characteristics of Strain FL19 of Genus Raoultella

### 1. Strain FL19 Promotes the Growth of PU Mycelium

PU mycelia were received from the National Resource Center for Chinese Materia Medica, China Academy of Chinese Medical Sciences, as documentedd in the non-patent literature “Pengjie Han, Tianrui Liu, Yuan Zheng, Ruiqi Song, Tiegui Nan, Xiaolong Yang, Luqi Huang and Yuan Yuan. A Mycorrhizal Bacteria Strain Isolated From PU Exhibits Broad-Spectrum Antifungal Activity. Front Plant Sci. 2022 Jul. 18: 13:954160. doi: 10.3389/fpls.2022.954160.eCollection 2022.”. This biological material was available to the public from the National Resource Center for Chinese Materia Medica, China Academy of Chinese Medical Sciences, and it is intended soley for the purpose of repeating relevant experiments of the present disclosure, and cannot be used for other purposes.

PU medium: 20 g of glucose, 5 g of yeast powder, 1 g of potassium dihydrogen phosphate, 1 g of magnesium sulfate and 15 g of agar were autoclaved at 121° C. for 15 min.

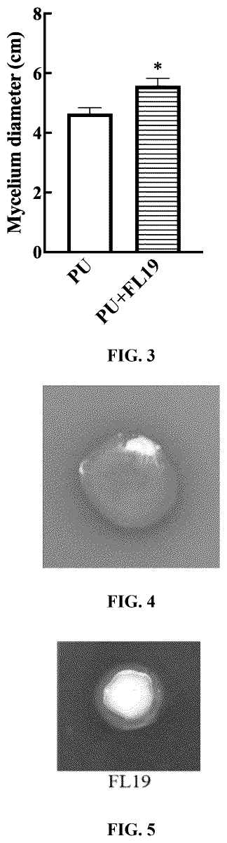

PU mycelia were co-cultured with strain FL19 using plate confrontation to observe the effect of strain FL19 on the growth of PU mycelium. The specific method was as follows. Firstly the PU mycelia were inoculated on a PU culture plate, and cultured for 15 d in the dark. A mycelium cake with a diameter of 6 mm was made on the edge of the PU colony plate by a driller to obtain a PU mycelium cake. A single colony of FL19 was picked and cultured in LB medium overnight until the OD₆₀₀ of the bacterial liquid was about 0.5 to obtain a strain FL19 inoculating liquid. The PU mycelium cake was placed in the center of a PU solid medium plate with a diameter of 90 mm, the strain FL19 inoculating liquid (experimental group, i.e., FL19 group) was plated at a position 2 cm away from the cake in a streaking manner, and the plate into which only the PU mycelium cake was inoculated was used as a control (control group), with a total of 6 replicates. Culture was performed at 25° C. in the dark for 15 d, and the diameter of the PU mycelium that grew for 15 d was measured by a crossover method. The diameter of the PU mycelium cake was measured. The results are as shown in FIG. 3, where in the control group, the PU mycelium cake had a mean diameter of 4.65 cm, and in the experimental group, the PU mycelium cake had a mean diameter of 5.58 cm (* indicates P<0.05). As can be seen, inoculating strain FL19 can promote the growth of PU mycelium.

### 2. Analysis of Growth-Promoting Characteristics of Strain FL19

### 2.1 Preparation of Suspension of Strain FL19

A single colony of strain FL19 was inoculated into LB medium and cultured at 25° C. for 1 d until the OD₆₀₀ of the bacterial liquid was 0.5 to obtain a suspension of strain FL19.

### 2.2 Phosphate Solubilization

Preparation of Pikovskaya (PVK) phosphate solubilizing medium: inorganic phosphorus solid medium (PVK medium) was purchased from Coolaber Science & Technology Co., Ltd. (catalog number MM5051). A total of 31.46 g of PVK powder was weighed and added to 1000 mL of deionized water. The pH was adjusted to 7.4±0.2. The mixture was autoclaved at 121° C. for 15 min, mixed evenly, and then poured into plates.

The growth-promoting strain was analyzed for phosphate solubilization using the PVK phosphate solubilizing medium. The specific method was as follows: 2 μL of the suspension of strain FL19 (OD₆₀₀=0.5) was taken, inoculated into a PVK phosphate solubilizing test medium by point inoculation, and cultured at 25° C. in the dark for 7 d. The phosphate solubilizing effect of the strain was confirmed by observing the presence of transparent phosphate solubilizing ring formed around the colony.

It was shown that a white transparent ring formed around strain FL19 (FIG. 4), indicating that the strain has the phosphate solubilizing effect.

### 2.3 Siderophore Production

CAS (chrome azurol S) medium: (1) Formula of CAS test plate: CAS dye contained 1 mmol/L CAS, 0.1 mmol/L FeCl₃, 4 mmol/L hexadecyl trimethyl ammonium bromide (HDTMA), and 0.1 mol/L phosphate buffer (pH=6.0); 100 mL of liquid contained 2.427 g of disodium hydrogen phosphate (Na₂HPO₄·12H₂O), 0.5905 g of sodium dihydrogen phosphate (NaH₂PO₄·2H₂O), 0.075 g of KH₂PO₄, 0.250 g of NH₄Cl, and 0.125 g of sodium chloride, and was diluted 10 times with sterile water when used. (2) Preparation of CAS test plate: 100 mL of liquid contained 1 mL of 20% sucrose solution, 3 mL of 10% acid hydrolyzed casein, 100 μL of 1 mmol/L CaCl₂), 2 mL of 1 mmol/L MgSO₄ and 1.8 g of agar, 5 mL of a salt solution and 5 mL of the CAS dye were slowly added at about 60° C. to obtain a blue test medium.

Strain FL19 was analyzed for siderophore production using the CAS medium. Two microliters of the suspension of strain FL19 (OD₆₀₀=0.5) was taken, inoculated into a CAS (chromeazurol) solid medium by point inoculation, and cultured at 25° C. in the dark for 7 d. The siderophore production was confirmed by observing the presence of a yellow halo around the colony.

It was shown that a yellow halo formed around strain FL19, indicating that the strain has the ability to produce siderophore (FIG. 5).

### 2.4 Auxin Production (IAA, Indole-3-Acetic Acid)

Preparation of Salkowski colorimetric liquid: 1 mL of 0.5 mol/L FeCl₃ was added to 50 mL of 35% perchloric acid.

The qualitative analysis of IAA production of the growth-promoting strain was performed using Salkowski colorimetric method. A single colony of strain FL19 was inoculated into LB liquid medium containing L-tryptophan (the content of L-tryptophan being 1.0 g/L) and cultured at 25° C. and 200 rpm for 1 d. The fermentation broth was centrifuged at 8000 rpm for 10 min, 1 mL of the supernatant was taken into a test tube, 2 mL of Salkowski colorimetric liquid was added, and the resulting mixture was uniformly mixed, and left to stand for 30 min in the dark. With LB liquid fermentation medium that does not contain L-tryptophan as a control, whether the strain had the ability to produce IAA was determined by observing whether the color of the solution turned red, where the darker the red color, the higher the IAA yield.

Preparation of IAA standard curve: The IAA standard was taken to prepare an IAA mother liquor with a concentration of 100 mg/L, which was then diluted separately to obtain: standard solutions with concentrations of 0.5, 1, 2, 5, 10, 15, 20, 25, 30, 35, 40, 45, 50, 60, 80 mg/L. An equal volume of Salkowski colorimetric liquid was added, the resulting mixture was reacted at room temperature in the dark for 30 min, with distilled water mixed with the Salkowski colorimetric liquid as a blank control. The absorbance at a wavelength 530 nm was determined using a microplate reader, with 3 replicates for each concentration gradient. A standard curve was plotted by taking the standard solution concentration as the abscissa and taking the OD 530 value corresponding to each standard solution concentration gradient as the ordinate. Y=0.0068X+0.0407, R²=0.9994.

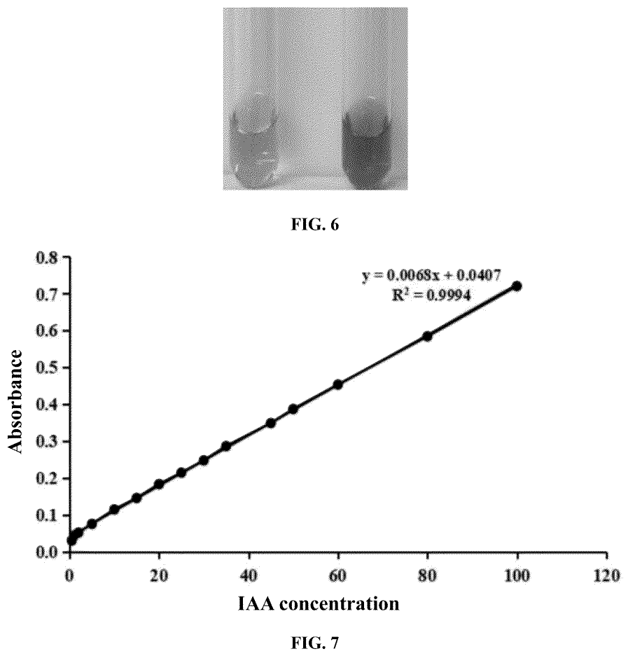

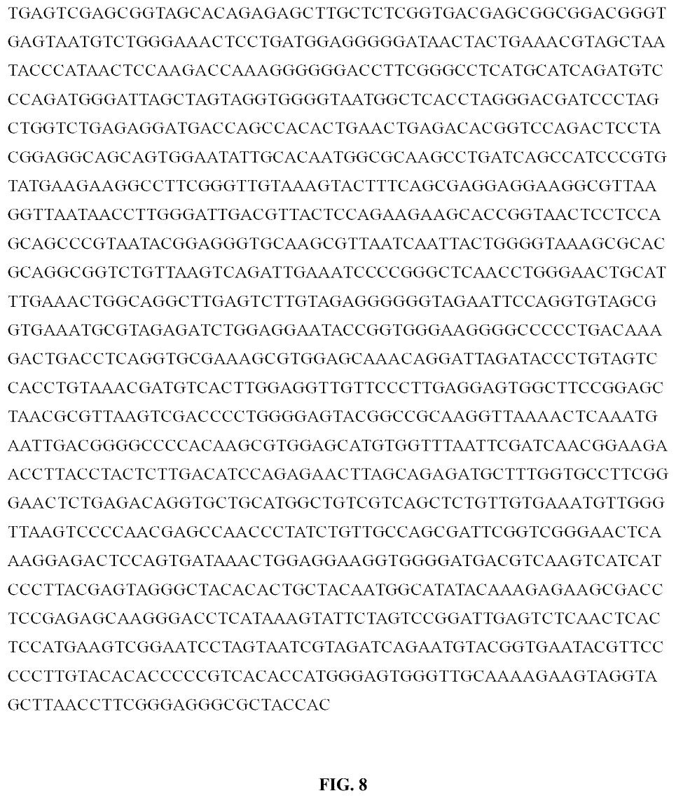

Determination of IAA content: FL19 strain was inoculated into LB liquid medium and cultured at 25° C. for 24 h. One milligram of the fermentation broth was centrifuged at 8000 r/min for 10 min, 100 μL of the supernatant and an equal amount of Salkowski colorimetric liquid were added into an ELISA plate, and the absorbance OD₅₃₀ of the strain was determined using a microplate reader. The obtained absorbance of the supernatant of FL19 strain was substituted into the IAA standard curve to obtain the IAA yield of the strain. The IAA yield of FL19 strain was calculated to be 73.7 mg/L, suggesting that strain FL19 can synthesize auxin IAA using L-tryptophan (FIG. 6 and FIG. 7).

The above provides a detailed description of the present disclosure. For those skilled in the art, it is understood that the invention can be implemented within a broad range of equivalent parameters, concentrations, and conditions without departing from the spirit and scope of the present disclosure and without the need for unnecessary experimentation. While specific embodiments of the present disclosure have been provided, it should be understood that further modifications may be made to the disclosure. In summary, in accordance with the principles of the disclosure, the application seeks to encompass any alterations, uses, or improvements to the invention, including those that fall outside the explicitly disclosed scope of this application, as long as they are carried out using conventional techniques known in the field.

What is claimed is:

1. A method for promoting growth of Polyporus umbellatus (PU) mycelium, wherein the method comprises administering to the PU mycelium a microbial agent comprising Raoultella ornithinolytica (R. ornithinolytica), wherein the R. ornithinolytica is a R. ornithinolytica strain having a strain number of FL19, and deposited under CGMCC No. 29044 in the China General Microbiological Culture Collection Center.

2. The method of claim 1, wherein the microbial agent further comprises a culture of the R. ornithinolytica, the culture being a substance obtained by culturing the R. ornithinolytica in a microorganism medium.

3. The method of claim 1, wherein the microbial agent further comprises metabolites of the R. ornithinolytica.

4. The method of claim 1, wherein the R. ornithinolytica strain produces siderophore;

5. The method of claim 1, wherein the R. ornithinolytica strain produces auxin.

6. The method of claim 1, wherein the R. ornithinolytica strain solublizes phosphate.


SEQUENCE LISTING (6 sequences)

SEQ ID NO: 1 - DNA, 1340 bp, synthetic construct
  source 1..1340: mol_type = other DNA; note = 16S rDNA of FL19
tgagtcgagcggtagcacagagagcttgctctcggtgacgagcggcggacgggtgagtaatgtctgggaaactcctgatggagggggataactactgaaacgtagctaatacccataactccaagaccaaaggggggaccttcgggcctcatgcatcagatgtcccagatgggattagctagtaggtggggtaatggctcacctagggacgatccctagctggtctgagaggatgaccagccacactgaactgagacacggtccagactcctacggaggcagcagtggaatattgcacaatggcgcaagcctgatcagccatcccgtgtatgaagaaggccttcgggttgtaaagtactttcagcgaggaggaaggcgttaaggttaataaccttgggattgacgttactccagaagaagcaccggtaactcctccagcagcccgtaatacggagggtgcaagcgttaatcaattactggggtaaagcgcacgcaggcggtctgttaagtcagattgaaatccccgggctcaacctgggaactgcatttgaaactggcaggcttgagtcttgtagaggggggtagaattccaggtgtagcggtgaaatgcgtagagatctggaggaataccggtgggaaggggccccctgacaaagactgacctcaggtgcgaaagcgtggagcaaacaggattagataccctgtagtccacctgtaaacgatgtcacttggaggttgttcccttgaggagtggcttccggagctaacgcgttaagtcgacccctggggagtacggccgcaaggttaaaactcaaatgaattgacggggccccacaagcgtggagcatgtggtttaattcgatcaacggaagaaccttacctactcttgacatccagagaacttagcagagatgctttggtgccttcgggaactctgagacaggtgctgcatggctgtcgtcagctctgttgtgaaatgttgggttaagtccccaacgagccaaccctatctgttgccagcgattcggtcgggaactcaaaggagactccagtgataaactggaggaaggtggggatgacgtcaagtcatcatcccttacgagtagggctacacactgctacaatggcatatacaaagagaagcgacctccgagagcaagggacctcataaagtattctagtccggattgagtctcaactcactccatgaagtcggaatcctagtaatcgtagatcagaatgtacggtgaatacgttcccccttgtacacacccccgtcacaccatgggagtgggttgcaaaagaagtaggtagcttaaccttcgggagggcgctaccac

SEQ ID NO: 2 - DNA, 796 bp, synthetic construct
  source 1..796: mol_type = other DNA; note = gyrB of FL19
actcctataagtctccggtggtctgcacggcgtgggcgtctctgtcgttaacgccctgtcacagaaactggagctggtgattcagccgataacaagattcacctcagatctatcccacgcgtacccaggcaccctgccgtcacctgataccgaaaaaacggtaccatggtgcgtttctggccgagctatgaaaccttcactaactcatcgaattgaatacgaaatcctggcaaaaccctcgtgagctgtcattcctaactcaggggtgtctatcccctccacaagcgtgatgcaaagaagaccatttccactacgaaggggcatcaaggcgttcttgagtatctcaacaagaacaaaacgccgatccacccgaatatcttctactcccacgaaaaagacggtattggctagaagtccgctgcagtggaacgatggtttccaggagaacatctactctttaccaacaacattccacaccgacgcggtacgcacctggcgggcttcctgcggcgatgacccgtaccctaacgcctatatggataaagagggttacagcaaaaaagcgaaggtcagcgccacccgacgatgccgtgaaggcctattgcctggtttccgtgaaggttccggatccgaagttctcctcacagaccaaagacaaactgtctcctccgaggtgaagacggcggtagagcagcagatgaacgaactctgaatgaatatctctggaaaaccctctgacccaaaatctgttggcaaaattattgatgcggcccgtgcgcgtgaagccgcgcgtcgt

SEQ ID NO: 3 - DNA, 20 bp, synthetic construct
  source 1..20: mol_type = other DNA; note = Primer 27F
agagtttgatcctggctcag

SEQ ID NO: 4 - DNA, 22 bp, synthetic construct
  source 1..22: mol_type = other DNA; note = Primer 1492R
tacggctaccttgttacgactt

SEQ ID NO: 5 - DNA, 20 bp, synthetic construct
  source 1..20: mol_type = other DNA; note = Primer VP-F
aacgcgttgtcwgaraaagt

SEQ ID NO: 6 - DNA, 20 bp, synthetic construct
  source 1..20: mol_type = other DNA; note = Primer VP-R
cctttacghcgrgtcatttc